(19) United States
(12) Patent Application Publication
(10) Pub. No.: US 2009/0118821 A1
(43) Pub. Date: May 7, 2009

(54) ENDOPROSTHESIS WITH POROUS RESERVOIR AND NON-POLYMER DIFFUSION LAYER

(75) Inventors: Torsten Scheuermann, Munich (DE); Jan Weber, Maastricht (NL); John T. Clarke, Kiniska (IE)

Correspondence Address: FISH & RICHARDSON PC, P.O. BOX 1022, MINNEAPOLIS, MN, 55440-1022, US

(73) Assignee: Boston Scientific Scimed, Inc., Maple Grove, MN (US)

(21) Appl. No.: 11/934,353

(22) Filed: Nov. 2, 2007

(51) Int. Cl. A61F 2/06

(52) U.S. Cl. 623/1.49; 623/1.42; 623/1.15

(57) ABSTRACT

An endoprosthesis such as a coronary stent includes a porous reservoir of drug, e.g., a porous layer formed of a ceramic and an overlayer formed of ceramic or metal for controlling elution of drug from the reservoir.

### TECHNICAL FIELD

This disclosure relates to endoprostheses with a porous reservoir and non-polymer diffusion layer.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire contents of which are hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

Passageways containing endoprostheses can become re-occluded. Re-occlusion of such passageways is known as restenosis. It has been observed that certain drugs can inhibit the onset of restenosis when the drug is contained in the endoprosthesis. It is sometimes desirable for an endoprosthesis-contained therapeutic agent, or drug to elute into the body fluid in a predetermined manner once the endoprosthesis is implanted.

### SUMMARY

In an aspect, the invention features an endoprosthesis having a surface, a first porous layer formed of a first ceramic over the surface, and a second porous layer formed of a second ceramic or a metal over the first layer.

In another aspect, the invention features a method of forming an endoprosthesis that includes forming a first porous layer on the endoprosthesis, introducing a drug into the first porous layer, and forming a second porous layer of a ceramic or a metal over the drug-containing first layer.

In another aspect, the invention features a method of forming an endoprosthesis that includes forming a first porous ceramic layer on the endoprosthesis by providing metal oxide sol and treating by heat application at a temperature of about 300° C. or less, and forming a second porous layer over the first layer without heating at a temperature of about 300° C. or more.

In another aspect, the invention features a method that includes providing a stent including a first porous layer formed of a ceramic, where the first porous layer incorporates a drug and the stent further includes a second porous layer of a different ceramic, and delivering the stent into the body.

Embodiments may include one or more of the following features. The first porous layer can include a drug. The second porous layer can have a different porosity than the first porous layer. The second layer can have a smaller porosity than the first layer. The pore size of the second layer can be smaller than the pore size of the first layer. The thickness of the second layer can be about 10 to about 500 nm. The thickness of the first layer can be about 0.1 to about 3 micron. The surface can be the surface of a stent body. The second layer can be formed of metal. The first layer and the second layer can form a drug delivery system substantially free of polymer. The second layer can be formed of a second ceramic. The second ceramic can be IROX. The second ceramic can have a smooth globular morphology. The second ceramic can be different from the first ceramic.

Embodiments may also include one or more of the following features. The first porous layer can be formed by a sol-gel process. The first porous layer can be formed by a nanocluster deposition process. The first porous layer can be formed of metal nanoclusters. The drug can be introduced by coating, dipping, or spraying in a solvent or applying substantially dry drug particles to the first porous layer. The drug can be introduced by pulse laser deposition (“PLD”). The second porous layer can be formed by PLD. The pores in the second layer can be formed by laser irradiation. The second layer can be a metal. The second layer can be a ceramic. The second layer can be IROX. The drug can be introduced during the sol-gel process. The second porous layer can be formed by a nanocluster deposition process.

Embodiments may include one or more of the following features. The drug can be incorporated in the metal oxide sol prior to heat treating. The metal oxide can be TiOx.

Embodiments may include one or more of the following advantages. Stents can be formed with high loadings of drug in a first porous ceramic coating (e.g., a drug reservoir) on select portions, such as the abluminal surface, and the drug delivery profile can be carefully controlled using an overlayer (e.g., a second layer) of a ceramic or a metal, without the use of a polymer. The drug can be loaded in large amount into the first porous coating on the stent. The first coating can have a high porosity, large pore openings, and large void cavities which can accommodate substantial amount of drug and can be relatively easily loaded by solvent techniques such as dipping or spraying, or direct dry loading of the drug into the pores or void regions of the first coating. The drug can be delivered to the first coating before the overlayer is provided, such that the drug can be delivered directly into the void regions without having to pass through the pores of the overlayer. The overlayer can be formed of a ceramic, e.g. IROX, which can have therapeutic advantages such as reducing the likelihood of restenosis and enhancing endothelialization. The morphology of the ceramic can be controlled to tune the therapeutic properties and the porosity of the overlayer to provide a desired drug release profile over an extended period. The overlayer can be a ceramic or metal that is compatible with the first porous coating of the stent. For example, the overlayer can be formed of the same ceramic as the first ceramic, which enhances bonding, biocompatibility, and reduces likelihood of degradation through corrosion. The porosity of the overlayer can be carefully controlled, e.g. the pore size can also be controlled by laser drilling such that a desired drug elution profile results over a long period of time. The overlayer can be formed by low temperature deposition process, such as PLD, which reduce the likelihood of degradation of drug previously provided in the void regions of the first coating. The first coating can be highly porous for accommodating a large quantity of drug and at the same time relatively thin. Likewise, the overlayer can be relatively thin, so as not to substantially increase the overall thickness of the stent wall. A polymer carrier can be avoided, which reduces the likelihood of polymer delamination and facilitates deployment from a delivery device during deployment.

Still further aspects, features, embodiments, and advantages follow.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIGS. 3A-3C are cross-sectional views of a stent wall.

FIG. 4 is a cross-sectional schematic of drug elution.

FIG. 5 is a flow diagram illustrating manufacture of a stent.

FIG. 6 is a schematic of a PLD system.

FIGS. 7A-7C are FESEM images: FIG. 7A and 7B are enlarged plan views of a stent wall surface, FIG. 7C is an enlarged cross-sectional view of a stent wall surface.

FIGS. 8A-8C are schematic views of ceramic morphologies.

FIG. 9 is an SEM image of a porous TiOₓ surface.

### DETAILED DESCRIPTION

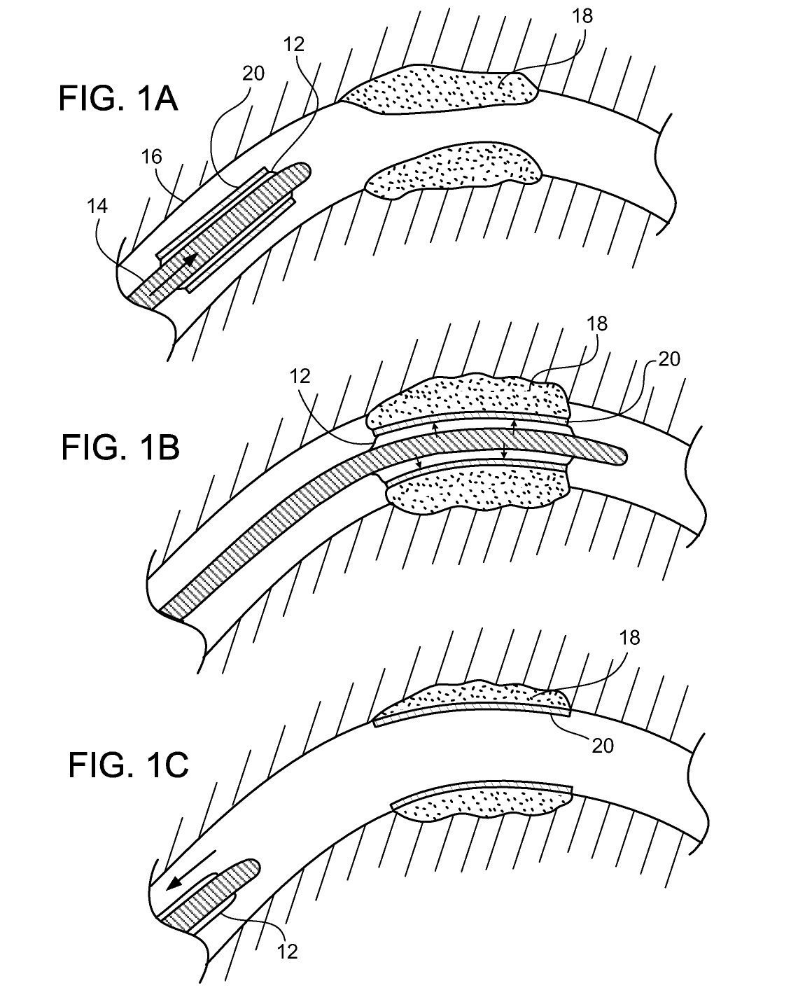

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

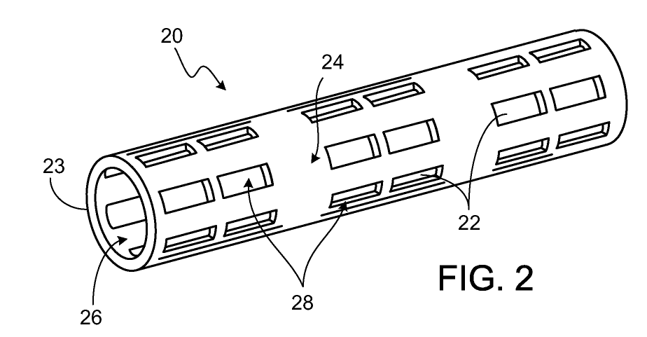

Referring to FIG. 2, the stent 20 includes a plurality of fenestrations 22 defined in a wall 23. Stent 20 includes several surface regions, including an outer, or abluminal, surface 24, an inner, adluminal, surface 26, and a plurality of cutface surfaces 28. The stent can be balloon expandable, as illustrated above, or a self-expanding stent. Examples of stents are described in Heath '721, supra.

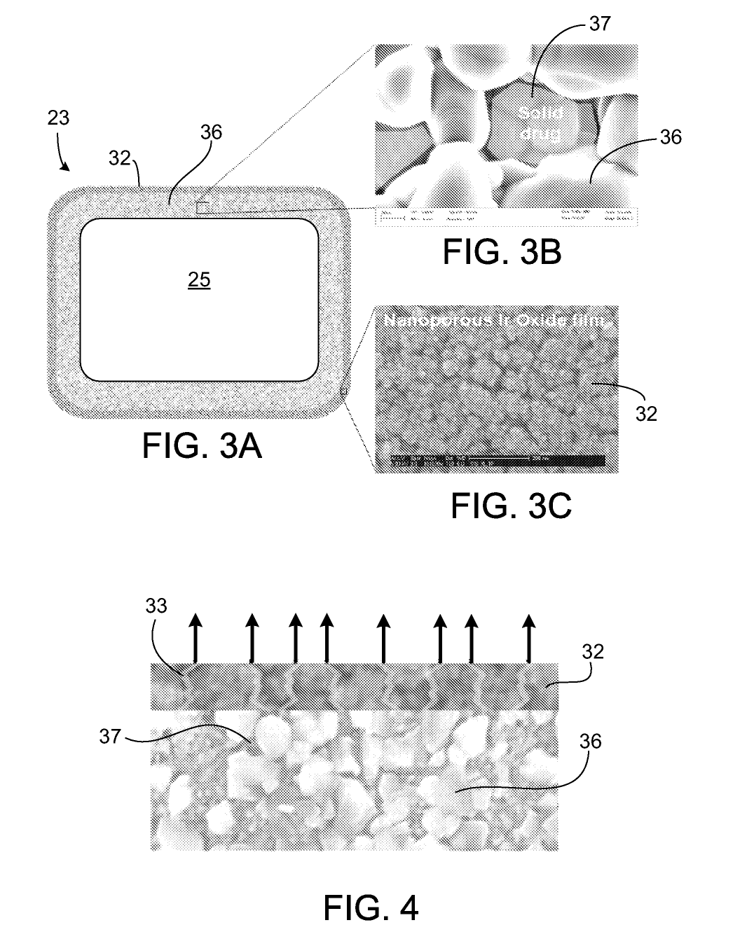

Referring to FIG. 3A, a cross-sectional view, a stent wall 23 includes a stent body 25 formed, e.g. of a metal or a polymer, and includes a first layer 36 formed of a first porous ceramic on the abluminal, adluminal, and cutface sides. A second layer 32, formed, e.g. of a second ceramic or a metal, covers the first layer 36. Referring to FIG. 3B, the first porous ceramic 36 has pores or void regions in which a drug 37 is stored. Referring to FIG. 3C, the ceramic or metal layer 32 is also porous, but with generally smaller pores than those of the first layer. Referring as well to FIG. 4, the ceramic or metal layer 32 with small pores 33 modulates the diffusion of drug from the first porous ceramic 36 to provide a desired release profile.

The first porous ceramic layer can be formed with high porosity (e.g., volume fraction of void space in the material) and large void regions which can accommodate large volumes of drug, without premature release of excessive doses of drug because the second ceramic or metal layer modulates the drug release profile. Moreover, the high porosity and large void areas accommodate a substantial amount of drug, such that the first porous layer is relatively thin and thus does not substantially increase the overall thickness of the stent wall. In embodiments, the first porous layer or the drug reservoir is deposited on a surface of a stent body by, e.g., a sol-gel reaction followed by a treatment process such as sintering, heat treatment, or water vapor treatment. In other embodiments, the first layer can be deposited by physical vapor deposition (“PVD”) processes. In particular embodiments, the porosity ratio (the ratio of the void volume to metal volume) is about 1:2, or more, e.g. about 1:1 or more, e.g. about 3:2. The drug loading per stent surface area (assuming a drug density of about 1 mg/mm³ and the first layer thickness of about 5 μm) is about 1.25 μg/mm² or more, e.g. about 2.5 μg/mm² or more, e.g. about 4 μg/mm². The pore or void diameter (or width) is in the range of about 0.1 to 5 microns (“μm”), e.g., about 0.5 to 3 microns. The thickness of the first layer is about three times the size of the pore diameter or less, e.g. about 0.3 to 15 microns, preferably about 0.5 to 5 microns. The second layer formed of a ceramic or metal is selected for compatibility for the first layer and to have a controlled drug elution and therapeutic properties. In embodiments, the second layer has a pore diameter (or width) of about 1 to 30 nm, e.g., about 1 to 20 nm, and a thickness of about 10 to 500 nm. In embodiments, the porosity ratio is selected to be about 10% to about 80%, e.g., about 10% to about 40%.

In embodiments, the porous coatings 32 and/or 36 can be formed of a ceramic or ceramics, such as iridium oxide (“IROX”), titanium oxide (“TiOₓ”), silicon oxide (“silica”) or oxides of niobium (“Nb”), tantalum (“Ta”), ruthenium (“Ru”) or mixture thereof. Certain ceramics, e.g. oxides, can reduce restenosis through the catalytic reduction of hydrogen peroxide and other precursors to smooth muscle cell proliferation.

The oxides can also encourage endothelial growth to enhance endothelialization of the stent. When a stent, is introduced into a biological environment (e.g., in vivo), one of the initial responses of the human body to the implantation of a stent, particularly into the blood vessels, is the activation of leukocytes, white blood cells which are one of the constituent elements of the circulating blood system. This activation causes an increase of reactive oxygen compound production. One of the species released in this process is hydrogen peroxide, H₂O₂, which is released by neutrophil granulocytes, which constitute one of the many types of leukocytes. The presence of H₂O₂ may increase proliferation of smooth muscle cells and compromise endothelial cell function, stimulating the expression of surface binding proteins which enhance the attachment of more inflammatory cells. A ceramic, such as IROX can catalytically reduce H₂O₂. The morphology of the ceramic can enhance the catalytic effect and reduce proliferation of smooth muscle cells. In a particular embodiment, IROX is selected to form the coating 32, which can have therapeutic benefits such as enhancing endothelialization while TiOₓ is selected to form 30 coating 36, which can have a desirable porous structure to accommodate large volumes of drug. TiOₓ are known to be blood-compatible, as described in Tsyganov et al., Surf Coat. Tech. 200:1041-44, 2005. Blood compatible substances show only minor induction of blood clot formation. Titanium oxide-based surfaces may also promote endothelial cell adhesion, which, in turn, may reduce thrombogenicity of stents delivered to blood vessels, as disclosed in Chen et al., Surf Coat. Tech. 186:270-76, 2004. IROX and other ceramics are discussed further in Alt et al., U.S. Pat. No. 5,980,566 and U.S. Ser. No. 10/651,562 filed Aug. 29, 2003. In particular embodiments, the first inner layer is TiOx and the second outer layer is IROX, e.g. having a desired morphology as described below. The TiOx structure has an average pore size of about 0.1 to 0.5 μm in diameter. The IROX layer has a thickness of about 10 to 500 nm and a pore size of about 1-20 nm, and a solid to cavity ratio of about 1:1.

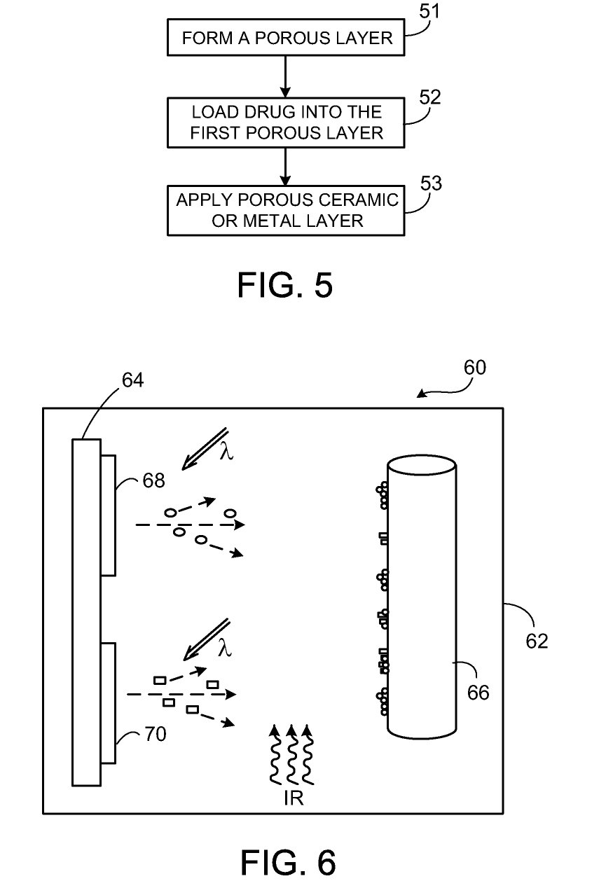

Referring to FIG. 5, the stent is formed by first providing the first porous ceramic layer on the stent (step 51). Next, a drug is delivered into the voids of the first layer (step 52). Finally, a second layer or the ceramic or metal overlayer is provided over the first porous layer (step 53) by a technique that uses low temperature to reduce the likelihood of damaging the drug or the porous region, such as pulsed layer deposition (“PLD”).

Referring particularly to step 51 in FIG. 5, the first porous ceramic layer can be deposited using a number of techniques including a sol-gel process or soluble oxides process. The sol-gel process is a versatile solution process for making ceramic and glass materials. In general, the sol-gel process involves the transition of a system from a liquid “sol” (mostly colloidal) into a solid “gel” phase. The starting materials or precursors used in the preparation of the sol are usually inorganic metal salts or metal organic compounds such as metal alkoxides. In a typical sol-gel process, the precursor is subjected to a series of hydrolysis and/or polymerization reactions to form a colloidal suspension, or a sol. Further processing of the sol enables one to make ceramic materials in different forms. For example, thin films can be produced on a piece of substrate (e.g., a stent or pre-stent such as metal tube) by spin coating, roll coating, spin coating, inkjet printing or spraying it with the sol. To obtain selective coating, e.g., coating of the abluminal surface only, instead of using dip coating within a solution, sol can be printed on the desired surface of the stent. When the colloidal particles in sol condense in a new phase, a wet gel, in which a solid macromolecule is immersed in a solvent, will form. With further drying and heat-treatment, the gel is converted into dense ceramic films. In general, sol-gel-derived ceramic porous layers are generated with use of an organic template or a surfactant as a template which needs to be removed at high temperatures, such as polyethylene glycol (PEG), polyvinylpyrrolidone (PVP), polyelectrolyte materials and oil emulsions.

In embodiments, as discussed, the porous layer formed in step 51 can include titanium oxide (“TiOₓ”), e.g., TiO₂. The precursor in the process therefore can be titanium-based, e.g., titanium (IV) bis(ammonium lactate) dihydroxide (TALH), or titanium alkoxide such as titanium (IV) butoxide (Ti(OBu)₄) or titanium tetraisopropoxide (TTIP). In a particular embodiment, within a sol-gel reaction, the precursor TTIP is mixed with an organic solvent, i.e. ethanol, and a controlled amount of PEG and water is dropped into the precursor solution therefore the sol-gel precursor hydrolyzes to form a titania sol by the presence of water molecules. The sol is then applied to a substrate to form a film or a coating. Afterwards, the coating is dried and the organic template (PEG) is removed by calcination at a high temperature, e.g., 400° C. or higher. Removal of the organic template leaves pores or voids in the overall structure where the organic template had been and allows the desired porosity. Changing template contents can generate coatings with different pore sizes, thus allowing generation of a desired drug release profile. For example, PEG of higher molecular weight leaves larger pores. In other embodiments, the first porous layer can be other oxides, such as iridium oxide (IROX) and silica; or a combination of TiOₓ and IROX; or a combination of TiOₓ and ruthenium oxide (RuOₓ); or a combination of TiOₓ, IROX and RuOₓ. Examples of sol-gel process are provided, e.g., in Manoharan et al., Proceedings of SPIE 3937: 44-50, 2000 and Guo et al., Surface & Coating Technology 198:24-29, 2005.

In certain embodiments, if high-temperature processing step (e.g., removing organic templates by heating at about 400° C.) is undesirable (e.g., if a metallic stent already has a coating of heat sensitive elements, such as certain polymers, drugs, or if the stent substrate is made out of a polymer), water vapor treatment at relatively low temperature, e.g., about 60 to 180° C., is used to generate the ceramic layer. To improve the crystallinity and mechanical properties with the exposure to water vapor, silica sol can be introduced into the TiOₓ sol to form a porous TiOₓ-silica layer, as described in Imai et al., J. Am. Ceram. Soc. 82:2301-2304, 1999. In further embodiments, with low-temperature processes, a drug can be embedded in a ceramic layer by mixing the drug with a sol-gel solution or applying the drug between sol deposition steps before the ceramic layer crystallizes or solidifies by the low temperature treatment. Optionally, the drug-incorporated ceramic layer can have an over coating with smaller pores to regulate drug release over an extended period of time. The over coating can be formed by, e.g., the low-temperature sol-gel process as discussed, or a PLD process, or nanocluster deposition process, which will be discussed in more detail below.

Referring to FIG. 5, step 52, a biologically active substance, e.g., a drug is loaded into the void regions of the first layer or the drug reservoir. In embodiments, the drug is loaded prior to forming the ceramic or metal overlayer, which facilitates loading because the drug does not have to diffuse through the ceramic or metal overlayer to reach the void regions of the underlying first layer. In addition, the high porosity and large cavity size of the first layer facilitate loading. In embodiments, the drug is loaded into void regions by dip coating or spraying the stent in a drug saturated solvent and drying under low temperature, e.g. ambient conditions. The drug is as a result precipitated into the void regions. The loading can be facilitated by repeatedly dipping and drying while the stent substrate is cooled under evacuated conditions. In embodiments, loading can also be facilitated by treating the first ceramic layer by corona discharge to make the surface more lipophilic, which attracts more lipophilic drugs to the first layer. Moreover, if the first layer is formed of TiOₓ, the hydrophilicity or hydrophobicity of the layer can be selected accordingly to facilitate drug loading. Stents coated with TiOₓ and methods of coating stents with TiOₓ are described in the U.S. Patent Application No. 60/818,101, filed Jun. 29, 2006. As described therein, coating stent with various combination of hydrophobic and/or hydrophilic TiOₓ allows for placing various biologically active substances on selected regions of the stent. Following application of TiOₓ coating, the medical device, e.g., a stent, can be exposed to conditions (e.g., UV light illumination) sufficient to cause desired regions of the device bearing TiOₓ coating to become hydrophilic or hydrophobic.

In embodiments, the drug is applied to the void regions as a dry powder of small particles. The particles can be blown with a high velocity gas jet such as air, inert gas (e.g. Argon and Nitrogen), or Carbon dioxide jet deep into the voids. The stent can be treated by dip coating to further load the voids. In embodiments, the drug particles are about 1 micron or less at their largest dimension, e.g. 500 nm or less. Suitable small particles, e.g. of paclitaxel, are available from Pharmasol GMBH, Blohmst 66 A, 12307 Berlin, Germany. In embodiments, the drug is applied to the first layer by a vapor deposition process, such as pulsed laser deposition. The drug can be deposited by providing drug as a target material in the PLD apparatus, as will be described further below. In embodiments, about 25% or more, e.g. about 50 to 90% of the void volume of the void regions in the first layer is occupied by drug after loading. The surface of the first layer can be cleaned by exposure to a gas stream, e.g. flowed horizontally over the surface, to remove drug on the outermost regions so that the ceramic or metal overlayer can be deposited directly onto the surfaces of the first layer to enhance layer adhesion and uniformity.

Referring to FIG. 5, step 53, in embodiments, the ceramic or metal overlayer is provided over the first porous coating by a technique that uses low temperature to reduce the likelihood of damaging the drug and/or the first coating, such as PLD. Referring to FIG. 6, the PLD system 60 includes a chamber 62 in which is provided a target assembly 64 and a stent substrate 66, such as a stent body or a pre-stent structure such as a metal tube. The target assembly includes a first target material 68, such as a ceramic (e.g., IROX), or a precursor to a ceramic (e.g., iridium metal), or a metal, e.g. stainless steel and a second target material 70, such as a drug. Laser energy (double arrows) is selectively directed onto the target materials to cause the target materials to be ablated or sputtered from the target assembly. The sputtered material is imparted with kinetic energy in the ablation process such that the material is transported within the chamber (single arrows) and deposited on the stent 66. In addition, the temperature of the deposited material can be controlled by heating, e.g. using an infrared source (squiggly arrows).

The pore size of the ceramic or metal overlayer can be controlled by varying the film thickness, the laser power, the total background pressure, and the partial pressure of oxygen, or the oxygen to argon ratio if reactive PLD is utilized. As discussed above, the drug can also be applied to the porous layer by PLD. For example, the second target material 70 can be formed of drug. Laser energy applied to the second target material can vaporize drug onto the first porous layer (the drug reservoir), and/or can vaporize drug with a ceramic or metal to form the overlayer or laser deposit a layer of drug onto the ceramic or metal overlayer.

The porosity of the ceramic can be controlled by selecting the morphology, crystallinity, thickness, and size of the clusters ablated and deposited. Higher crystallinity, more defined grain morphologies, and thinner coatings provide greater porosity. Higher crystallinity and more defined grain morphologies can be formed by heating the deposited ceramic. Coating thickness is controlled by controlling deposition time. Higher laser energies can provide larger cluster sizes.

In particular embodiments, the laser energy is produced by an excimer laser operating in the ultraviolet, e.g. at a wavelength of about 248 nm. The laser energy is about 100-700 mJ, the fluence is in the range of about 10 to 50 mJ/cm². The background pressure is in the range of about 1E-5 mbar to 1 mbar. The background gas includes oxygen. The substrate temperature is also controlled. The temperature of the substrate is between 25 to 300° C. during deposition. Substrate temperature can be controlled by directing an infrared beam onto the substrate during deposition using, e.g. a halogen source. The temperature is measured by mounting a heat sensor in the beam adjacent the substrate. The temperature can be varied to control the morphology of the ceramic material. The selective ablating of the ceramic or drug is controlled by mounting the target materials on a moving assembly that can alternately bring the materials into the path of the laser. Alternatively, a beam splitter and shutter can be used to alternatively or simultaneously expose multiple materials. PLD deposition services are available from Axyntec, Augsburg, Germany. Suitable ceramics include metal oxides and nitrides, such as of iridium, zirconium, titanium, hafnium, niobium, tantalum, ruthenium, platinum, and aluminum. In embodiments, the thickness of the coatings is in the range of about 50 nm to about 2 um, e.g. 100 nm to 500 nm. Pulsed laser deposition is also described in application U.S. Ser. No. 11/752,736, filed May 23, 2007 [Attorney Docket No. 10527-801001], and in Geretovszky et al., Thin Solid Films, 2004, 453-454, 245. In other embodiments, another physical vapor deposition (“PVD”) process is selected such as magnetron sputtering e.g. an iridium target under an oxygen atmosphere or an IROX target. Sputtering deposition is described in application U.S. Ser. No. 11/752,772, filed May 23, 2007 [Attorney Docket No. 10527-805001]. In the case of a ceramic or a metal over coating, the porosity can be further controlled by laser ablating apertures into the layer with, e.g. a U.V. laser.

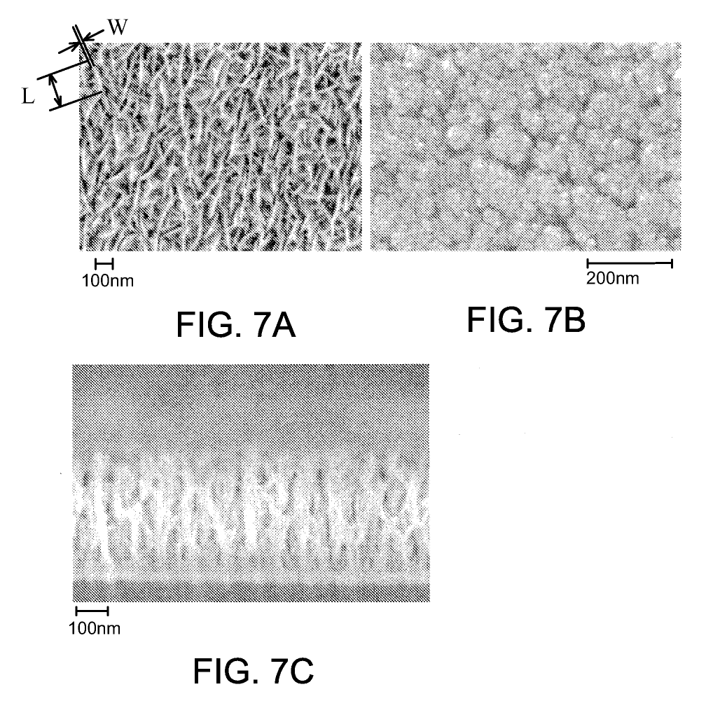

Referring to FIGS. 7A and 7B, the morphology of the ceramic can be varied between relatively rough surfaces and relatively smooth surfaces, which can each provide particular mechanical and therapeutic advantages, such as a controlled porosity to modulate drug release from the drug reservoir layer. Referring particularly to FIG. 7A, a ceramic coating can have a morphology characterized by defined grains and high roughness. Referring particularly to FIG. 7B, a ceramic coating can have a morphology characterized by a higher coverage, globular surface of generally lower roughness. The defined grain, high roughness morphology provides a high surface area characterized by crevices between and around spaced grains into which the polymer coating can be deposited and interlock to the surface, greatly enhancing adhesion. Defined grain morphologies also allow for greater freedom of motion and are less likely to fracture as the stent is flexed in use and thus the coating resists delamination of the ceramic from an underlying surface and reduces delamination of a possible overlaying coating. The stresses caused by flexure of the stent, during expansion or contraction of the stent or as the stent is delivered through a tortuously curved body lumen increase as a function of the distance from the stent axis. As a result, in embodiments, a morphology with defined grains is particularly desirable on abluminal regions of the stent or at other high stress points, such as the regions adjacent fenestrations which undergo greater flexure during expansion or contraction. Smoother globular surface morphology provides a surface which is tuned to facilitate endothelial growth by selection of its chemical composition and/or morphological features. Certain ceramics, e.g. oxides, can reduce restenosis through the catalytic reduction of hydrogen peroxide and other precursors to smooth muscle cell proliferation. The oxides can also encourage endothelial cell growth to enhance endothelialization of the stent. As discussed above, when a stent, is introduced into a biological environment (e.g., in vivo), one of the initial responses of the human body to the implantation of a stent, particularly into the blood vessels, is the activation of white blood cells. This activation causes a release of hydrogen peroxide, H₂O₂. The presence of H₂O₂ may increase proliferation of smooth muscle cells and compromise endothelial cell function, stimulating the expression of surface binding proteins which enhance the attachment of more inflammatory cells. A ceramic, such as IROX can catalytically reduce H₂O₂. The smoother globular surface morphology of the ceramic can enhance the catalytic effect and enhance growth of endothelial cells.

Referring particularly to FIG. 7C, a cross-sectional view of the ceramic layer with surface morphology shown in FIG. 7B, channels formed of interconnected pores in the ceramic are visible. In embodiments, the channels have a diameter selectively controlled by deposition parameters as discussed above. The bigger the diameter, the faster the drug release rate. In particular embodiments, the channel diameter is selected to be about 10 nm or less to control the drug release over a extended period of time.

The morphology of the ceramic is controlled by controlling the energy of the sputtered clusters on the stent substrate. Higher energies and higher temperatures result in defined grain, higher roughness surfaces. Higher energies are provided by increasing the temperature of the ceramic on the substrate, e.g. by heating the substrate or heating the ceramic with infrared radiation. In embodiments, defined grain morphologies are formed at temperatures of about 250° C. or greater. Globular morphologies are formed at lower temperatures, e.g. ambient temperatures without external factors. The heating enhances the formation of a more crystalline ceramic, which forms the grains. Intermediate morphologies are formed at intermediate values of these parameters. The composition of the ceramic can also be varied. For example, oxygen content can be increased by providing oxygen gas in the chamber.

The morphology of the surface of the ceramic is characterized by its visual appearance, its roughness, and/or the size and arrangement of particular morphological features such as local maxima. In embodiments, the surface is characterized by definable sub-micron sized grains. Referring particularly to FIG. 7A, for example, in embodiments, the grains have a length, L, of the of about 50 to 500 nm, e.g. about 100-300 nm, and a width, W, of about 5 nm to 50 nm, e.g. about 10-15 nm. The grains have an aspect ratio (length to width) of about 5:1 or more, e.g. 10:1 to 20:1. The grains overlap in one or more layers. The separation between grains can be about 1-50 nm. In particular embodiments, the grains resemble rice grains.

Referring particularly to FIG. 7B, in embodiments, the surface is characterized by a more continuous surface having a series of shallow globular features. The globular features are closely adjacent with a narrow minima between features. In embodiments, the surface resembles an orange peel. The diameter of the globular features is about 100 nm or less, and the depth of the minima, or the height of the maxima of the globular function is e.g. about 50 nm or less, e.g. about 20 nm or less. In other embodiments, the surface has characteristics between high aspect ratio definable grains and the more continuous globular surface and/or has a combination of these characteristics. For example, the morphology can include a substantially globular base layer and a relatively low density of defined grains. In other embodiments, the surface can include low aspect ratio, thin planar flakes. The morphology type is visible in FESEM images at 50 KX.

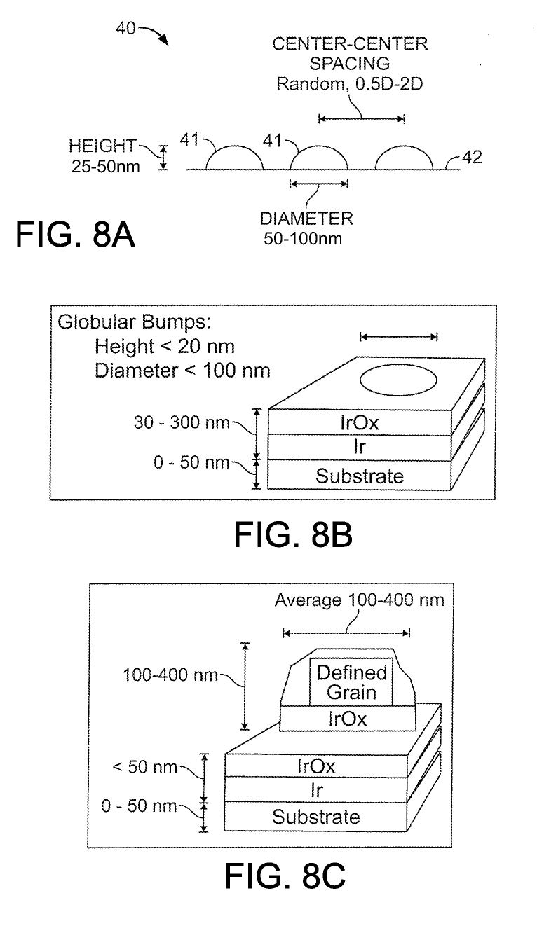

Referring to FIGS. 8A-8C, morphologies are also characterized by the size and arrangement of morphological features such as the spacing, height and width of local morphological maxima. Referring particularly to FIG. 8A, a coating 40 on a substrate 42 is characterized by the center-to-center distance and/or height, and/or diameter and/or density of local maxima. In particular embodiments, the average height, distance and diameter are in the range of about 400 nm or less, e.g. about 20-200 nm. In particular, the average center-to-center distance is about 0.5 to 2× the diameter.

Referring to FIG. 8B, in particular embodiments, the morphology type is a globular morphology, the width of local maxima is in the range of about 100 nm or less and the peak height is about 20 nm or less. In particular embodiments, the ceramic has a peak height of less than about 5 nm, e.g., about 1-5 nm, and/or a peak distance less than about 15 nm, e.g., about 10-15 nm. Referring to FIG. 8C, in embodiments, the morphology is defined as a grain type morphology. The width of local maxima is about 400 nm or less, e.g. about 100-400 nm, and the height of local maxima is about 400 nm or less, e.g. about 100-400 nm. As illustrated in FIGS. 8B and 8C, the select morphologies of the ceramic can be formed on a thin layer of substantially uniform, generally amorphous IROX, which is in turn formed on a layer of iridium metal, which is in turn deposited on a metal substrate, such as titanium or stainless steel. The spacing, height and width parameters can be calculated from AFM data.

The roughness of the surface is characterized by the average roughness, Sa, the root mean square roughness, Sq, and/or the developed interfacial area ratio, Sdr. The Sa and Sq parameters represent an overall measure of the texture of the surface. Sa and Sq are relatively insensitive in differentiating peaks, valleys and the spacing of the various texture features. Surfaces with different visual morphologies can have similar Sa and Sq values. For a surface type, the Sa and Sq parameters indicate significant deviations in the texture characteristics. Sdr is expressed as the percentage of additional surface area contributed by the texture as compared to an ideal plane the size of the measurement region. Sdr further differentiates surfaces of similar amplitudes and average roughness. Typically Sdr will increase with the spatial intricacy of the texture whether or not Sa changes.

In embodiments, the ceramic has a defined grain type morphology. The Sdr is about 30 or more, e.g. about 40 to 60. In addition or in the alternative, the morphology has an Sq of about 15 or more, e.g. about 20 to 30. In embodiments, the Sdr is about 100 or more and the Sq is about 15 or more. In other embodiments, the ceramic has a globular type surface morphology. The Sdr is about 20 or less, e.g. about 8 to 15. The Sq is about 15 or less, e.g. about less than 8 to 14. In still other embodiments, the ceramic has a morphology between the defined grain and the globular surface, and Sdr and Sq values between the ranges above, e.g. an Sdr of about 1 to 200 and/or an Sq of about 1 to 30. The Sa, Sq, and Sdr can be calculated from AFM data.

The morphology of the ceramic coating can exhibit high uniformity. The uniformity provides predictable, tuned therapeutic and mechanical performance of the ceramic. The uniformity of the morphology as characterized by Sa, Sq or Sdr and/or average peak spacing parameters can be within about ±20% or less, e.g. ±10% or less within a 1 μm square. In a given stent region, the uniformity is within about ±10%, e.g. about ±1%. For example, in embodiments, the ceramic exhibits high uniformity over an entire surface region of stent, such as the entire abluminal or adluminal surface, or a portion of a surface region, such as the center 25% or 50% of the surface region. The uniformity is expressed as standard deviation. Uniformity in a region of a stent can be determined by determining the average in five randomly chosen 1 μm square regions and calculating the standard deviation. Uniformity of a morphology type in a region is determined by inspection of FESEM data at 50 kx.

The ceramics are also characterized by surface composition, composition as a function of depth, and crystallinity. In particular, the amounts of oxygen or nitride in the ceramic is selected for a desired catalytic effect on, e.g., the reduction of H₂O₂ in biological processes. The composition of metal oxide or nitride ceramics can be determined as a ratio of the oxide or nitride to the base metal. In particular embodiments, the ratio is about 2 to 1 or greater, e.g. about 3 to 1 or greater, indicating high oxygen content of the surface. In other embodiments, the ratio is about 1 to 1 or less, e.g. about 1 to 2 or less, indicating a relatively low oxygen composition. In particular embodiments, low oxygen content globular morphologies are formed to enhance endothelialization. In other embodiments, high oxygen content defined grain morphologies are formed, e.g., to enhance adhesion and catalytic reduction. Composition can be determined by x-ray photoelectron spectroscopy (XPS). Depth studies are conducted by XPS after FAB sputtering. The crystalline nature of the ceramic can be characterized by crystal shapes as viewed in FESEM images, or Miller indices as determined by x-ray diffraction. In embodiments, defined grain morphologies have a Miller index of<101>. Globular materials have blended amorphous and crystalline phases that vary with oxygen content. Higher oxygen content typically indicates greater crystallinity. Further discussion of ceramics and ceramic morphology and computation of roughness parameters is provided in U.S. Ser. No. 11/752,772, U.S. Ser. No. 11/752,736, and appendices.

In certain embodiments, referring back to FIGS. 3A and 4, the second layer 32 can be a porous metallic coating. For example, by using a nanocluster deposition system available from, e.g., Mantis Deposition Ltd., England (http://www.mantisdeposition.com), ionized metal nanoparticles, such as copper, gold, ruthenium, stainless steel, can be produced by magnetron sputtering followed by thermalization and condensation in relatively high pressure zones (e.g., about 0.1 to a few millibars) and accelerated towards a substrate, e.g., a drug-containing ceramic layer, by an applied electric field. Most of the nanoparticles or nanoclusters generated are charged and may therefore be mass selected by a linear quadrupole or a mass filter and selectively deposited on the substrate. For example, typical diameter of the nanoparticles generated can be about 0.7 to 20 nm and size distribution of the nanoparticles can be selectively narrowed to about ±20%, or even to ±2%. The kinetic energy (e.g., about 10 eV to 10 keV) of the nanoparticles partly controlled by the applied electric field may induce particles melting upon impact the substrate and may cause damage to the substrate and/or the drug incorporated in the substrate. In embodiments, a first collection of particles are deposited with a kinetic energy, e.g., 200 eV, that is high enough for the particles to adhere to the drug-containing ceramic layer but substantially causes no heat damage to the drug incorporated in the ceramic. A second collection of particles with higher kinetic energy, e.g., 2 keV, can then be deposited on top of the first collection. In other embodiments, ceramic nanoparticles can also be generated by reactive sputtering and be deposited on the drug-containing ceramic layer using the nanocluster deposition system. In one particular embodiment, the stent can be formed by first shooting large (e.g., >100 nm) nanoclusters (ceramic or metallic) to a selected surface using the system discussed above to form a large-pore layer, which then can readily be filled with a drug. After loading the drug, an overlayer formed of small nanoclusters (e.g., <5 nm) which therefore has small pores and channels can be deposit over the large-pore layer by changing the filter setting for the desired particle size. One or more intermediate layers formed of medium-sized nanoclusters (e.g., about 25-50 nm) particles may be provided between the drug-containing layer and the small-pore overlayer. Nanoparticle deposition is further disclosed by Weber et al., Provisional Application No. 60/857,849 filed Nov. 9, 2006 [BSC Docket No. 06-01579], U.S. Ser. No. 11/860,253, filed Sep. 24, 2007 [Attorney Docket No. 10527-809001], A. H. Kean, Mantis Deposition Ltd., NSTI Nano Tech 2006, Boston, May 7-11, 2006, and in Y Qiang, Surface and Coating Technology, 100-101, 27-32 (1998). In embodiments, localized heating, e.g. by laser or particle bombardment can be used to fuse or sinter deposited particles to, e.g. enhance bonding, between first and second layers.

In embodiments, ceramic or metal is adhered only on the abluminal surface of the stent. This construction may be accomplished by, e.g. coating the stent before forming the fenestrations. In other embodiments, ceramic or metal is adhered only on abluminal and cutface surfaces of the stent. This construction may be accomplished by, e.g., coating a stent containing a mandrel, which shields the luminal surfaces. Masks can be used to shield portions of the stent. In embodiments, the stent metal can be stainless steel, chrome, nickel, cobalt, tantalum, superelastic alloys such as nitinol, cobalt chromium, MP35N, and other metals, e.g., biodegradable metals such as magnesium or its alloys, iron or its alloys, and tungsten or its alloys. In other embodiments, the stent can be formed of biodegradable or non-biodegradable polymers, such as poly(lactic-co-glycolic acid) (PLGA), Polylactic acid (PLLA), polyurethane, or their copolymer or mixture. The non-biodegradabledrug reservoir, e.g., TiO2, and the non-biodegradable drug-eluting regulatory membrane can remain embedded in the vessel tissue after degradation of the stent body. Suitable stent materials and stent designs are described in Heath '721, supra. In embodiments, the morphology and composition of the ceramic or metal are selected to enhance adhesion to a particular stent metal. For example, in embodiments, the ceramic is deposited directly onto the metal surface of a stent body, e.g. a stainless steel, without the presence of an intermediate metal layer. In other embodiments, a layer of metal common to the ceramic is deposited onto the stent body before deposition to the ceramic. For example, a layer of iridium may be deposited onto the stent body, followed by deposition of IROX onto the iridium layer. Other suitable ceramics include metal oxides and nitrides, such as of iridium, zirconium, titanium, hafnium, niobium, tantalum, ruthenium, platinum and aluminum. The ceramic can be crystalline, partly crystalline or amorphous. The ceramic can be formed entirely of inorganic materials or a blend of inorganic and organic material (e.g. a polymer). In other embodiments, the morphologies described herein can be formed of metal. As discussed above, different ceramic materials can be provided in different regions of a stent. For example, different materials may be provided on different stent surfaces. A rougher, defined grain material may be provided on the abluminal surface to, e.g. enhance adhesion while a material with globular features can be provided on the adluminal surface to enhance endothelialization. In embodiments, the drug is provided directly into the first porous coating without a polymer. In other embodiments, the drug is applied to the first porous coating with a polymer. Suitable polymers include, for example, copolymers thereof with vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers. Other suitable polymers are discussed in U.S. Ser. No. 11/752,736, filed May 23, 2007 [Attorney Docket No. 10527-801001]. The polymer is preferably capable of absorbing a substantial amount of drug solution. When applied as a coating on a medical device in accordance with the present invention, the dry polymer is typically on the order of from about 1 to about 50 microns thick, preferably about 1 to 10 microns thick, and more preferably about 2 to 5 microns. Very thin polymer coatings, e.g., of about 0.2-0.3 microns and much thicker coatings, e.g., more than 10 microns, are also possible. Multiple layers of polymer coating can be provided onto a medical device. Such multiple layers are of the same or different polymer materials.

### EXAMPLE

### Example 1

### Sol-Gel Method

Titanium tetraisopropoxide (TTIP) (97%, Aldrich), ethanol (99.5%, Aldrich), water, HCl (37%) and PEG were used as the starting materials. TTIP (1.25 ml) are added to a solution made up of 10 ml ethanol and 0.3 ml HCl at room temperature. After 1 h of stirring, a given volume of water and a controlled amount of PEG (HO(CH2CH2O))nH) (1 mol % of the TTIP) are dropped into the solution. Sol samples formed are applied to the stent substrate by dip/spray coating. The average molecular weight of PEG used is 2000. The coated stents are first dried at 60° C. and calcined at 550° C. for 1 h in air.

### Example 2

### Spin Coating

Metal coupons (e.g., stainless steel) are coated by spin coating three coatings of titanium (IV) trifluoroacetate in butanone (100 g/L, 0.3 cm3 per coating, initially spin @ 500 rpm for approximately 3 minutes, then increasing spin to 3000 ppm for approximately 3 minutes) onto the coupons. Each coating is annealed at 800° C. or lower temperature for e.g., two hours. The depth of the coatings can be controlled as required from a few hundred nanometers to a few tens of microns as required. The coatings prepared, as above, are estimated to have a thickness of about 5-10 micron based on SEM analysis of the coating that flaked around the edges of the coupons.

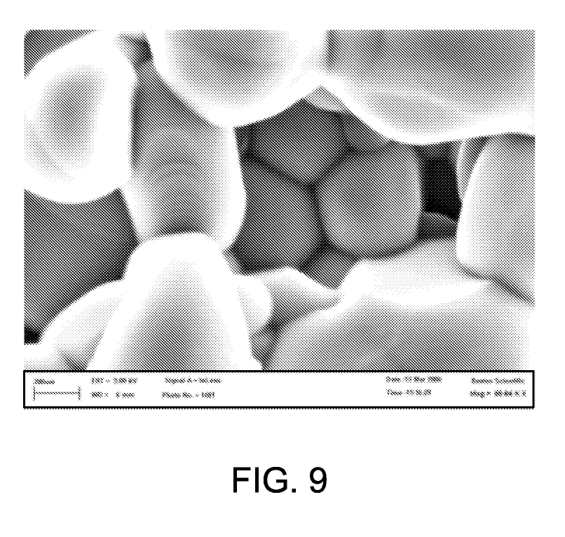

Referring to FIG. 9, a field emission scanning electron microscopy (“FESEM”) image of the TiOₓ surface (here soluble oxide), a highly porous structure is formed having surface openings greater than 500 nm and about a few microns deep.

The terms “therapeutic agent”, “pharmaceutically active agent”, “pharmaceutically active material”, “pharmaceutically active ingredient”, “biologically active substance ”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), immunosuppressants(e.g., everolimus, tacrolimus), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application Nos. 2005/0019265 and 2005/0251249. A functional molecule, e.g. an organic, drug, polymer, protein, DNA, and similar material can be incorporated into groves, pits, void spaces, and other features of the ceramic.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6A1-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, and urethral lumens.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprostheses or medical devices, such as catheters, guide wires, and filters.

All publications, patent applications, and patents, are incorporated by reference herein in their entirety.

Still other embodiments are in the following claims.

What is claimed is:

1. An endoprosthesis, comprising:
a surface,
a first porous layer formed of a first ceramic over the surface, and
a second porous layer formed of a second ceramic or a metal over the first layer.

2. The endoprosthesis of claim 1, wherein the first porous layer includes a drug.

3. The endoprosthesis of claim 2, wherein the second layer has less porosity than the first layer.

4. The endoprosthesis of claim 3, wherein the pore size of the second layer is smaller than the pore size of the first layer.

5. The endoprosthesis of claim 1, wherein the thickness of the second layer is about 10 to about 500 nm.

6. The endoprosthesis of claim 1, wherein the thickness of the first layer is about 0.1 to about 3 micron.

7. The endoprosthesis of claim 1, wherein the surface is the surface of a stent body.

8. The endoprosthesis of claim 1, wherein the second layer is formed of metal.

9. The endoprosthesis of claim 1, wherein the first layer and the second layer form a drug delivery system substantially free of polymer.

10. The endoprosthesis of claim 1, wherein the second layer is formed of a second ceramic.

11. The endoprosthesis of claim 10, wherein the second ceramic is IROX.

12. The endoprosthesis of claim 10, wherein the second ceramic has a smooth globular morphology.

13. The endoprosthesis of claim 12, wherein the second ceramic is different from the first ceramic.

14. A method of forming an endoprosthesis, comprising:
forming a first porous layer on the endoprosthesis,
introducing a drug into the first porous layer, and
forming a second porous layer of a ceramic or metal over the drug-containing first layer.

15. The method of claim 14, comprising forming the first porous layer by a sol-gel process.

16. The method of claim 14, comprising forming the first porous layer by a nanocluster deposition process.

17. The method of claim 16, comprising introducing the drug by coating, dipping, spraying in a solvent or applying substantially dry drug particles to the first porous layer.

18. The method of claim 14, comprising introducing the drug by PLD.

19. The method of claim 14, comprising forming the second porous layer by PLD.

20. The method of claim 14, comprising forming pores in the second layer by laser irradiation.

21. The method of claim 14, wherein the second layer is a metal.

22. The method of claim 14, wherein the second layer is ceramic.

23. The method of claim 22, wherein the second layer is IROX.

24. The method of claim 14, comprising introducing the drug during the sol-gel process.

25. The method of claim 14, comprising forming the second porous layer by a nanocluster deposition process.

26. A method of forming an endoprosthesis, comprising
forming a first porous ceramic layer on the endoprosthesis by providing metal oxide sol and treating by heat application at temperatures of about 300° C. or less; and
forming a second porous layer over the first layer without heating at a temperature of about 300° C. or more.

27. The method of claim 26, comprising incorporating a drug in the metal oxide sol prior to heat treating.

28. The method of claim 27, comprising the metal oxide is TiOx.

29. A method comprising
providing a stent including a first porous layer formed of a ceramic, the first porous layer incorporating a drug, the stent further including a second porous layer of a different ceramic, and delivering the stent into the body.
